(19) United States
(12) Patent Application Publication
(10) Pub. No.: US 2023/0033047 A1
(43) Pub. Date: Feb. 2, 2023

(54) Estrogen Metabolite Levels And Cancer Driver Gene Mutations In Lung Cancer Risk Stratification And Treatment

(71) Applicant: Institute For Cancer Research d/b/a The Research Institute of Fox Chase Cancer Center, Philadelphia, PA (US)

(72) Inventors: Margie L. Clapper, Philadelphia, PA (US); Joseph N. Bodor, Philadelphia, PA (US); Jing Peng, Philadelphia, PA (US); Joseph Treat, Philadelphia, PA (US)

(21) Appl. No.: 17/758,495

(22) PCT Filed: Jan. 11, 2021
(86) PCT No.: PCT/US21/12892

§ 371 Date: Jul. 7, 2022

Related U.S. Application Data
(60) Provisional application No. 62/959,914, filed on Jan. 11, 2020.

(51) Int. Cl. A61K 31/7076; A61K 31/09; A61K 31/4375; A61K 31/352; A61K 31/05; A61K 31/404; A61K 31/4196; A61K 31/566; A61P 35/00; A61P 11/00

(52) U.S. Cl. CPC A61K 31/7076; A61K 31/09; A61K 31/4375; A61K 31/352; A61K 31/05; A61K 31/404; A61K 31/4196; A61K 31/566; A61P 35/00; A61P 11/00

(57) ABSTRACT

The present disclosure provides methods of risk stratification for the development of lung cancer and/or lung cancer recurrence, methods of treatment of a human having lung cancer with therapeutic agents for preventing estrogen metabolite production, and methods of treating a human having a high risk of developing lung cancer with therapeutic agents for preventing estrogen metabolite production.

### REFERENCE TO GOVERNMENT GRANTS

This invention was made with government support under Grant No. CA217161 awarded by the National Institutes of Health. The government has certain rights in the invention.

### FIELD

The present disclosure is directed, in part, to methods of risk stratification for the development of lung cancer and/or lung cancer recurrence, and methods of treatment of a human having lung cancer or a high risk of developing lung cancer with therapeutic agents for preventing estrogen metabolite production.

### BACKGROUND

Lung cancer is the leading cause of cancer death among men and women in the U.S. In addition to cigarette smoke, estrogen exposure has been associated recently with lung cancer in women. The use of hormone replacement therapy has been related to both a younger age of diagnosis of lung cancer and decreased median survival. Metabolism and detoxification of the constituents of cigarette smoke also play a role in lung carcinogenesis. In addition, the activity and carcinogenicity of estrogen depend on the metabolic transformation of 17β-estradiol. It is believed that the balance between the activity of Phase I and II metabolism enzymes affects cell protection from carcinogens and plays an important role in lung carcinogenesis.

The outcome of patients with lung cancer varies widely depending on individual variables including tumor type, stage at presentation, smoking status, and gender. For instance, the 5-year survival of lung cancer patients who are current smokers is significantly lower than that of lung cancer patients who never smoked (16% and 23%, respectively, p=0.004). Recent pharmacogenetic studies have also found that polymorphisms in DNA repair enzymes impact the outcome of lung cancer patients treated with specific chemotherapeutic agents.

Considerable inter-individual genetic variability exists in the Phase I and II enzymes, with several studies suggesting that select polymorphisms are associated with an increased risk for lung cancer development. Nebert et al., Nat. Rev. Cancer, 2006, 6, 947-60. Members of the cytochrome P450 family 1, including CYP1A1 and CYP1B1, activate exogenous substances such as polycyclic aromatic hydrocarbons as well as endogenous substances such as estrogens to highly reactive intermediates. Phase II metabolism enzymes, including glutathione S-transferase M1 (GSTM1) and catechol-O-methyltransferase (COMT), are, in general, responsible for the conversion of these intermediates to inactive conjugates. Previous reports suggest that the replacement of isoleucine with valine at codon 462 of CYP1A1 (I462V) combined with deletion of the GSTM1 gene confer an increased risk of lung cancer in females (odds ratio (OR) 6.54; 95% confidence interval (95% CI) 1.07-40.00). Dresler et al., Lung Cancer, 2000, 30, 153-60. The I462V polymorphism leads to enhanced CYP1A1 enzyme activity, promoting carcinogen activation, while deletion of the GSTM1 gene impairs the capacity to conjugate and eliminate carcinogens.

CYP1B1 is the predominant enzyme that catalyzes the 4-hydroxylation of estrogen to yield a putative carcinogen. The hydroxylation activity of CYP1B1 is of particular importance since activated carcinogens induce DNA single-strand breakages and mutations. The cytochrome P450 1B1 metabolizes estradiol to 4-hydroxyestradiol (4-OHE), which is a catechol estrogen that has been shown to induce depurination of DNA that leads to DNA mutations. In addition to estrogens, CYP1B1 also bioactivates a range of chemically diverse procarcinogens including benzo[a]pyrene. Sequences of naturally occurring human CYP1B1 nucleic acid molecules are described in GenBank™ Accession (GB #U56438) and Tang et al., J. Biol. Chem., 1996, 271, 28324.

Epidermal growth factor receptor, also known as EGFR, ErbB1 and HER1, is a cell-surface receptor for members of the epidermal growth factor family of extracellular ligands. Alterations in EGFR activity have been implicated in certain cancers. EGFR is a member of the ErbB family of closely related receptors including EGFR (ErbB-1), Her2/neu (ErbB-2), Her3 (ErbB-3) and Her4 (ErbB-4). Activation of EGFR leads to receptor tyrosine kinase activation and a series of downstream signaling events that mediate increases in cellular proliferation, motility, adhesion, invasion, blocking of apoptosis and resistance to chemotherapy. EGFR and its ligands, EGF and transforming growth factor alpha, are expressed in over 80% of NSCLC. Upon ligand binding, EGFR homodimerizes or forms heterodimers with other members of the ErbB family leading to receptor phosphorylation and activation of downstream signaling events. EGFR activation leads to the association with multiple signaling mediators such as She, Grb2, Src, JAKs, PLD, PLCGAMMA, and PLCGAMMA, and PI3K and subsequently to the activation of signaling transducers such as ERK1/2, FAK, JNK, STATs, and Akt. The importance of EGFR in tumorigenesis has prompted the development and commercialization of therapeutic agents that block its function.

Thus, there is a need in the art to enhance the confidence in diagnostic information and/or risk stratification of humans having or suspected of having lung cancer, including the assessment of a human's risk of developing lung cancer or lung cancer recurrence, and the applicability of treating a human having a high risk of developing lung cancer with a therapeutic agent for preventing estrogen metabolite production.

### SUMMARY

The present disclosure provides methods for stratifying the risk of a human for developing a lung cancer event. The methods comprise performing or having performed an assay on a biological sample obtained from the human, whereby the assay determines the level of total estrogen in the biological sample. The methods also comprise performing or having performed an assay on a biological sample obtained from the human, whereby the assay determines the level of 4-OHEs and/or 2-OHEs in the biological sample. The methods also comprise determining the ratio of 4-OHEs to total estrogen, the ratio of 2-OHEs to total estrogen, and/or the ratio of 4-OHEs to 2-OHEs. In some embodiments, the methods also comprise performing or having performed an analysis of estrogen-induced mutations in one or more cancer driver genes in a biological sample obtained from the human. The risk of developing a lung cancer event is stratified based on the results of the total estrogen and estrogen metabolite assays and/or analysis of estrogen-induced mutations in one or more cancer driver genes. When the human has an equal or lower ratio of 4-OHEs to total estrogen, or an equal or lower ratio of 2-OHEs to total estrogen, or an equal or lower ratio of 4-OHEs to 2-OHEs compared to the same ratios from a cancer-free subject, then the human has a lower risk of developing the lung cancer event. When the human has a higher ratio of 4-OHEs to total estrogen, or a higher ratio of 2-OHEs to total estrogen, or a higher ratio of 4-OHEs to 2-OHEs compared to the same ratios from a cancer-free subject, then the human has a higher risk of developing the lung cancer event. When the human comprises an estrogen-induced mutation in the one or more cancer driver genes, then the human has a higher risk of developing the lung cancer event. In these methods, the lung cancer event is developing a lung cancer, having a lung cancer recurrence, and/or a lower survival rate.

In some embodiments, the level of 4-OHEs is the sum of 4-OHE₁ and 4-OHE₂. In some embodiments, the level of 2-OHEs is the sum of 2-OHE₁ and 2-OHE₂. In some embodiments, the total estrogen is the sum of E₁, E₂, E₃, 4-OHE₁, 4-OHE₂, 2-OHE₁, 2-OHE₂, 2-OME₁, and 2-OME₂. In some embodiments, the biological sample for determining the level of total estrogen, 4-OHEs, and/or 2-OHEs is urine, serum, or lung tissue.

In some embodiments, the biological sample for analysis of estrogen-induced mutations in one or more cancer driver genes is lung tissue or blood. In some embodiments, the cancer driver gene having the estrogen-induced mutation is EGFR, ALK, ROS1, RET, BRAF, HER2, DDR2, FGFR1, PDGFRA, KRAS, PIK3CA, PTEN, H3F3A, KDR, or MET. In some embodiments, the cancer driver gene having the estrogen-induced mutation is EGFR. In some embodiments, the EGFR gene comprises a deletion of exon 19 (ex19Del), a point mutation in exon 21, a mutation in exon 18, or a mutation in exon 20. In some embodiments, the EGFR gene comprises a G719S mutation, a G719C mutation, a G719A mutation, an L861Q mutation, an L858R mutation, an L845R mutation, a T790M mutation, a C797S mutation, an ex19Del mutation, or an ex20Ins mutation.

In some embodiments, the human is a tobacco smoker. In some embodiments, the human is a never-smoker. In some embodiments, the cancer is non-small cell lung cancer (NSCLC) or small cell lung cancer (SCLC). In some embodiments, the cancer is NSCLC.

In some embodiments, the methods further comprise performing or having performed an assay on a biological sample obtained from the human, whereby the assay determines the activity of one or more enzymes in the metabolism of estrogen, wherein when the human has a higher activity of one or more enzymes producing 2-OHEs or 4-OHEs compared to a cancer-free subject, then the human has higher risk of developing the lung cancer event, and when the human has the same or a lower activity of one or more enzymes producing 2-OHEs or 4-OHEs compared to a cancer-free subject, then the human has a lower risk of developing the lung cancer event. In some embodiments, the one or more enzymes in the metabolism of estrogen is chosen from CYP1B1, CYP1A1, and COMT.

In some embodiments, the methods further comprise: i) administering to the human having a higher ratio of 4-OHEs to total estrogen, or a higher ratio of 2-OHEs to total estrogen, or a higher ratio of 4-OHEs to 2-OHEs a therapeutic agent that inhibits CYP1B1, a CYP1A1/CYP1B1 dual inhibitor, a therapeutic agent that induces or restores COMT, and/or a therapeutic agent that inhibits estrogen production; and/or ii) administering to the human having an estrogen-induced mutation in the one or more cancer driver genes a therapeutic agent that inhibits the production or activity of the cancer driver gene product. In some embodiments, the therapeutic agent that inhibits CYP1B1 is 2,3′,4,5′-tetramethoxystilbene, berberine, homoeriodictyol, or transresveratrol. In some embodiments, the therapeutic agent that induces COMT is a divalent metal cation, S-adenosylmethionine, indole-3-carcinol, diindolylmethane, or a phytoestrogen. In some embodiments, the therapeutic agent that inhibits estrogen production is anastrozole, exemestane, or letrozole. In some embodiments, the therapeutic agent that inhibits the production or activity of the cancer driver gene product is a tyrosine kinase inhibitor. In some embodiments, the tyrosine kinase inhibitor is erlotinib, gefitinib, afatinib, osimertinib, or dacomitinib, or any combination thereof. In some embodiments, the human is administered a combination of a tyrosine kinase inhibitor and one or more of a therapeutic agent that inhibits CYP1B1, a CYP1A1/CYP1B1 dual inhibitor, a therapeutic agent that induces or restores COMT, or a therapeutic agent that inhibits estrogen production. In some embodiments, the human is administered surgery, radiation therapy, proton therapy, ablation therapy, hormone therapy, chemotherapy, immunotherapy, stem cell therapy, follow up testing, diet management, vitamin supplementation, nutritional supplementation, exercise regimen, physical therapy, a prosthetic, transplantation, reconstruction, psychological counseling, social counseling, education, or regimen compliance management, or any combination thereof.

The present disclosure also provides methods of treating a human having a high risk of developing a lung cancer event but who does not yet have lung cancer, the method comprising administering to the human a therapeutic agent that inhibits CYP1B1, a CYP1A1/CYP1B1 dual inhibitor, a therapeutic agent that induces or restores COMT, or a therapeutic agent that inhibits estrogen production. In some embodiments, the therapeutic agent that inhibits CYP1B1 is 2,3′,4,5′-tetramethoxystilbene, berberine, homoeriodictyol, or transresveratrol. In some embodiments, the therapeutic agent that induces COMT is a divalent metal cation, S-adenosylmethionine, indole-3-carcinol, diindolylmethane, or a phytoestrogen. In some embodiments, the therapeutic agent that inhibits estrogen production is anastrozole, exemestane, or letrozole. In some embodiments, the methods further comprise administering to the human a tyrosine kinase inhibitor. In some embodiments, the tyrosine kinase inhibitor is erlotinib, gefitinib, afatinib, osimertinib, or dacomitinib, or any combination thereof. In some embodiments, the human has an EGFR mutation.

### BRIEF DESCRIPTION OF THE DRAWINGS

FIG. 1 shows CYP1-mediated metabolism of estrogen in the lung.

FIG. 2 shows an example of anchorage independent growth of colonies of HBEC-57KT cells resulting from transformation by 4-OHE₂ and E₂. Colonies were stained with crystal violet.

FIG. 3 shows the ability of E2, 4-OHE₁, 4-OHE₂, 2-OHE₁, and 2-OHE₂ to transform HBEC-57KT cells at concentrations of 2, 10, 50, and 100 nM. Cells were exposed to the estrogen species for 27 weeks and assayed for anchorage independent growth. Values represent the mean number of colonies (3 technical replicates) present following 5 weeks of growth in soft agar.

FIG. 4 shows a comet assay demonstrating that estrogens are able to induce DNA damage in select HBEC-KT cell lines. E₂ and 4-OHE₂ induced DNA damage in HBEC-KT cells with high-activity CYP1B1 and low-activity COMT (HBEC-57KT and -65KT). Values=fold difference of comet tail moment in E₂/4-OHE vs. DMSO-treated cells (set as 1) (mean±SEM). P≤0.05 (*); 0.01 (**) 0.001 (***) by t-test.

FIGS. 5A, 5B, and 5C show the ratio of 4-OHEs to total estrogen, the ratio of 2-OHEs to total estrogen, and the ratio of level of 4-OHEs to 2-OHEs from EGFR- and ALK-mutated NSCLC patients and cancer-free subjects. Box plots denote the medians (values labeled) and the 75^{th} and 25^{th} percentiles. The “whiskers” represent the most extreme points (≤1.5 times the inter-quartile range). Outliers are points outside the box. P-values were calculated using the Wilcoxon-rank sum test (*P<0.10, **P<0.05).

### DESCRIPTION OF EMBODIMENTS

The terminology used herein is for the purpose of describing particular embodiments only and is not intended to be limiting. Unless otherwise expressly stated, it is in no way intended that any method or aspect set forth herein be construed as requiring that its steps be performed in a specific order. Accordingly, where a method claim does not specifically state in the claims or descriptions that the steps are to be limited to a specific order, it is in no way intended that an order be inferred, in any respect. This holds for any possible non-expressed basis for interpretation, including matters of logic with respect to arrangement of steps or operational flow, plain meaning derived from grammatical organization or punctuation, or the number or type of aspects described in the specification.

As used herein, the singular forms “a,” “an,” and “the,” include plural referents unless expressly stated otherwise.

As used herein, the terms “administration” and “administering” to the human refers to providing a compound to the subject by any administration route.

As used herein, the term “nucleic acid molecule” includes any chain of at least two nucleotides, which may be unmodified or modified RNA or DNA, hybrids of RNA and DNA, and may be single, double, or triple stranded. Nucleic acid molecules include any one or more of genomic DNA, mRNA, or cDNA produced from the mRNA.

As used herein, the term “comprising” may be replaced with “consisting” or “consisting essentially of” in particular embodiments as desired.

As used herein, the terms “treat”, “treating”, and “treatment” refer to eliciting the desired biological response, such as a therapeutic and/or prophylactic effect. In some embodiments, a therapeutic effect comprises one or more of a decrease/reduction in a lung cancer, a decrease/reduction in the severity of a lung cancer (such as, for example, a reduction or inhibition of development or a lung cancer), a decrease/reduction in symptoms and lung cancer-related effects, delaying the onset of symptoms and lung cancer-related effects, reducing the severity of symptoms of lung cancer-related effects, reducing the severity of an acute episode, reducing the number of symptoms and lung cancer-related effects, reducing the latency of symptoms and lung cancer-related effects, an amelioration of symptoms and lung cancer-related effects, reducing secondary symptoms, reducing secondary infections, preventing relapse to a lung cancer, decreasing the number or frequency of relapse episodes, increasing latency between symptomatic episodes, increasing time to sustained progression, expediting remission, inducing remission, augmenting remission, speeding recovery, or increasing efficacy of or decreasing resistance to alternative therapeutics, and/or an increased survival time of the affected host animal, following administration of the agent or composition comprising the agent. A prophylactic effect may comprise a complete or partial avoidance/inhibition or a delay of a lung cancer development/progression (such as, for example, a complete or partial avoidance/inhibition or a delay), and an increased survival time of the affected host animal, following administration of a therapeutic protocol. Treatment of a lung cancer encompasses the treatment of patients already diagnosed as having or suspected of having any form of lung cancer at any clinical stage or manifestation, and the delay of the onset or evolution or aggravation or deterioration of the symptoms or signs of a lung cancer. Treatment of a human also encompasses the treatment of patients who have not yet developed a lung cancer but who have a high risk of developing a lung cancer.

It has been observed in accordance with the present disclosure that levels of particular estrogen metabolites in human lung tissue may correlate with an increased risk of developing lung cancer. Without intending to be limited to any particular theory or mechanism, it is believed that the greater relative level of CYP1B1-generated estrogen metabolite 4-OHEs compared to CYP1A1-generated estrogen metabolite 2-OHEs in NSCLC patients may contribute to the development of lung tumors. This, in turn, suggests that targeting CYP1B1, the enzyme responsible for production of 4-OHEs, may be of therapeutic interest. Thus, estrogen metabolite levels, particularly 4-OHEs, may be useful for stratifying the risk of a human for developing lung cancer. In addition, estrogen-induced mutations in cancer driver genes may also be useful for stratifying the risk of a human for developing lung cancer. Further, estrogen-induced mutations in cancer driver genes may also be useful for designing a treatment protocol prior to tumor development. Accordingly, the present disclosure provides methods for stratifying the risk of a human having particular estrogen metabolite levels and/or estrogen-induced mutations in cancer driver genes for developing lung cancer, assessing the likelihood of a human having particular estrogen metabolite levels and/or estrogen-induced mutations in cancer driver genes for responding to particular treatments prior to development of lung cancer, and methods of treatment of a human having particular estrogen metabolite levels and/or estrogen-induced mutations in cancer driver genes and who have not yet developed lung cancer.

2-OHEs can also transform normal lung cells. These metabolites are generated mostly by CYP1A1 and, to a minor extent, by 1B1. CYP1A1 is a minor contributor to 4-OHE production as well. In addition, high levels of 2-OHEs may also be indicative a higher risk of developing a lung cancer event.

The present disclosure provides methods for stratifying the risk of a human for developing a lung cancer event. In some embodiments, the methods comprise performing or having performed an assay on a biological sample obtained from the human, whereby the assay determines the level of total estrogen in the biological sample. In some embodiments, the methods also comprise performing or having performed an assay on a biological sample obtained from the human, whereby the assay determines the level of estrogen metabolites, such as 4-OHEs (i.e., 4-OHE₁ and/or 4-OHE₂) and/or 2-OHEs (i.e., 2-OHE₁ and/or 2-OHE₂) in the biological sample. The methods also comprise determining the ratio of 4-OHEs to total estrogen, the ratio of 2-OHEs to total estrogen, and/or the ratio of 4-OHEs to 2-OHEs. The risk of developing the lung cancer event is stratified depending upon the results achieved for the total estrogen and estrogen metabolite assays. When the human has an equal or lower ratio of 4-OHEs to total estrogen, or an equal or lower ratio of 2-OHEs to total estrogen, or an equal or lower ratio of 4-OHEs to 2-OHEs compared to the same ratios from a cancer-free subject, then the human has a lower risk of developing the lung cancer event. When the human has a higher ratio of 4-OHEs to total estrogen, or a higher ratio of 2-OHEs to total estrogen, or a higher ratio of 4-OHEs to 2-OHEs compared to the same ratios from a cancer-free subject, then the human has a higher risk of developing the lung cancer event. In some embodiments, the methods also comprise performing or having performed an analysis of estrogen-induced mutations in one or more cancer driver genes (as opposed to a mutational analysis of an established tumor) in a biological sample obtained from the human. When the human comprises an estrogen-induced mutation in one or more cancer driver genes, then the human has a higher risk of developing a lung cancer event. In the methods described herein, the lung cancer event is developing a lung cancer, having a lung cancer recurrence, and/or having a lower survival rate. Thus, the ratio of 4-OHEs to total estrogen, the ratio of 2-OHEs to total estrogen, the ratio of 4-OHEs to 2-OHEs, and/or the presence of an estrogen-induced mutation in one or more cancer driver genes in the human are factors that can be used for stratifying the risk of developing a lung cancer event and for designing a treatment regimen for humans who have not yet developed a lung cancer event but who have a high risk of developing a lung cancer event.

In any of the methods described herein, the lung cancer event is any one or more of developing lung cancer, lung cancer recurrence, and/or having a lower survival rate. In some embodiments, the lung cancer event is a higher risk of developing lung cancer. In some embodiments, the lung cancer event is an increased risk of lung cancer recurrence. In some embodiments, the lung cancer event is having a lower survival rate.

The methods described herein comprise performing or having performed an assay on a biological sample obtained from the human, whereby the assay determines the level of total estrogen in the biological sample. In some embodiments, the total estrogen is the sum of estrone (E₁), estradiol (E₂), estriol (E₃), 4-OHE₁, 4-OHE₂, 2-OHE₁, 2-OHE₂, 2-OME₁, and 2-OME₂. In some embodiments, the total estrogen also includes 4-OME₁ and 4-OME₂. In some embodiments, the determined concentration of total estrogens is compared to one or more estrogen reference concentrations for a healthy subject (i.e., a cancer-free subject), estrogen reference concentrations for a subject at risk for developing a lung cancer event, or estrogen reference concentrations for a subject having a lung cancer event. In some embodiments, the determined concentration of total estrogens is compared to one or more estrogen reference concentrations for a healthy subject (i.e., a cancer-free subject). In some embodiments, the determined concentration of total estrogens is compared to one or more estrogen reference concentrations for a subject at risk for developing a lung cancer event. In some embodiments, the determined concentration of total estrogens is compared to one or more estrogen reference concentrations for a subject having a lung cancer event.

The methods described herein also comprise performing or having performed an assay on a biological sample obtained from the human, whereby the assay determines the level of 4-OHEs (i.e., 4-OHE₁ and/or 4-OHE₂) and/or 2-OHEs (i.e., 2-OHE₁ and/or 2-OHE₂) in the biological sample. In some embodiments, the 4-OHE estrogen metabolite is 4-OHE₂. In some embodiments, the 2-OHE estrogen metabolite is 2-OHE₂. In some embodiments, the determined concentration of estrogen metabolites is compared to one or more estrogen metabolite reference concentrations for a healthy subject (i.e., a cancer-free subject), estrogen metabolite reference concentrations for a subject at risk for developing a lung cancer event, or estrogen metabolite reference concentrations for a subject having a lung cancer event. In some embodiments, the determined concentration of estrogen metabolites is compared to one or more estrogen metabolite reference concentrations for a healthy subject (i.e., a cancer-free subject). In some embodiments, the determined concentration of estrogen metabolites is compared to one or more estrogen metabolite reference concentrations for a subject at risk for developing a lung cancer event. In some embodiments, the determined concentration of estrogen metabolites is compared to one or more estrogen metabolite reference concentrations for a subject having a lung cancer event. The assay may also determine parent estrogens and other estrogen metabolites as well, and constitutes total estrogen.

In any of the methods described herein, the biological sample for determining the level of total estrogen, 4-OHEs, and/or 2-OHEs is any fluid or tissue in which estrogen and/or estrogen metabolites can be found, and in which concentrations of each can be determined. For example, the biological sample can be lung tissue or tissue from the aerodigestive tract. In some embodiments, the biological sample can be buccal tissue. In some embodiments, the biological sample can be blood. In some embodiments, the biological sample can be intrathoracic tissue or cells obtained from the bronchus or lung, or can comprise extrathoracic tissue from the mouth or nose. In some embodiments, the biological sample can be a biologic fluid, including blood, mucus, sputum, urine, saliva, and tears. In some embodiments, the biological sample for determining the level of total estrogen, 4-OHEs, and/or 2-OHEs is urine, serum, or lung tissue. The biological samples can be obtained according to any suitable technique.

The methods described herein also comprise determining the ratio of 4-OHEs to total estrogen (4-OHEs/total estrogen), the ratio of 2-OHEs to total estrogen (2-OHEs/total estrogen), and/or the ratio of 4-OHEs to 2-OHEs (4-OHEs/2-OHEs). In some embodiments, the ratio of 4-OHEs/total estrogen is determined. In some embodiments, the ratio of 2-OHEs/total estrogen is determined. In some embodiments, the ratio of 4-OHEs/2-OHEs is determined. The determination of the ratios comprises determining the ratios of concentrations of the one or more estrogens and one or more estrogen metabolites. In some embodiments, the determined ratio is compared to the ratio for a healthy subject (i.e., a cancer-free subject), the ratio for a subject at risk for developing a lung cancer event, or the ratio for a subject having a lung cancer event. In some embodiments, the determined ratio is compared to the ratio for a healthy subject (i.e., a cancer-free subject). In some embodiments, the determined ratio is compared to the ratio for a subject at risk for developing a lung cancer event. In some embodiments, the determined ratio is compared to the ratio for a subject having a lung cancer event.

The risk of developing the lung cancer event is stratified depending upon the results achieved for the total estrogen and estrogen metabolite assays and/or analysis of estrogen-induced mutations in one or more cancer driver genes. For example, when the human has an equal or lower ratio of 4-OHEs to total estrogen, or an equal or lower ratio of 2-OHEs to total estrogen, or an equal or lower ratio of 4-OHEs to 2-OHEs compared to the same ratios from a cancer-free subject, then the human has a lower risk of developing the lung cancer event. In addition, when the human has a higher ratio of 4-OHEs to total estrogen, or a higher ratio of 2-OHEs to total estrogen, or a higher ratio of 4-OHEs to 2-OHEs compared to the same ratios from a cancer-free subject, then the human has a higher risk of developing the lung cancer event.

The methods described herein also comprise performing or having performed an analysis of estrogen-induced mutations in one or more cancer driver genes (as opposed to a mutational analysis of an established tumor) in a biological sample obtained from the human. When the human comprises an estrogen-induced mutation in the one or more cancer driver genes, then the human has an increased risk of developing a lung cancer event. In some embodiments, the analysis of estrogen-induced mutations comprises determining the nucleotide sequence of the one or more cancer driver genes in the biological sample obtained from the human and comparing it to the nucleotide sequence of a reference or wild type nucleic acid sequence for the one or more cancer driver genes. The comparison can be carried out, for example, using a processor programmed to compare nucleic acid sequences. In some embodiments, the mutational analysis comprises a hybridization assay. A hybridization assay can be carried out in vitro, and can be carried out using a support such as an array. For example, nucleic acid molecules (genomic DNA, mRNA, etc.) obtained from the human can be labeled and contacted with an array of probes affixed to a support. The probes can be specific for estrogen-induced mutations in a cancer driver gene.

The biological sample for determining the analysis of estrogen-induced mutations in one or more cancer driver genes can be any tissue in which the nucleic acid sequence encoding the cancer driver gene can be obtained. Examples of such tissues include, but are not limited to, blood and lung tissue. In some embodiments, the biological sample for determining the analysis of estrogen-induced mutations in one or more cancer driver genes is lung tissue. In some embodiments, the biological sample for determining the analysis of estrogen-induced mutations in one or more cancer driver genes is blood. In some embodiments, the biological sample for determining the analysis of estrogen-induced mutations in one or more cancer driver genes is tissue from the aerodigestive tract. In some embodiments, the biological sample for determining the analysis of estrogen-induced mutations in one or more cancer driver genes is buccal tissue. In some embodiments, the biological sample for determining the analysis of estrogen-induced mutations in one or more cancer driver genes is intrathoracic tissue or cells obtained from the bronchus or lung, or can comprise extrathoracic tissue from the mouth or nose. In some embodiments, the biological sample for determining the analysis of estrogen-induced mutations in one or more cancer driver genes is a biologic fluid, including blood, serum, mucus, sputum, urine, saliva, and tears. The tissue can be a fresh isolate, or frozen, or can be fixed, including a formalin-fixed tissue. The tissue from which the nucleotide sequence encoding the cancer driver gene is obtained can be the same tissue or different tissue from which the estrogen and estrogen metabolites are obtained. The sequence of the nucleic acid molecule can be determined using any sequencing method suitable in the art.

The cancer driver gene can be any of the cancer driver genes associated with lung cancer. In some embodiments, the cancer driver gene is chosen from epidermal growth factor receptor (EGFR), ALK, ROS1, RET, BRAF, HER2, DDR2, FGFR1, PDGFRA, KRAS, PIK3CA, PTEN, H3F3A, KDR, and MET. In some embodiments, the cancer driver gene is EGFR. In some embodiments, the cancer driver gene is ALK. In some embodiments, the cancer driver gene is ROS1. In some embodiments, the cancer driver gene is RET. In some embodiments, the cancer driver gene is BRAF. In some embodiments, the cancer driver gene is HER2. In some embodiments, the cancer driver gene is DDR2. In some embodiments, the cancer driver gene is FGFR1. In some embodiments, the cancer driver gene is PDGFRA. In some embodiments, the cancer driver gene is KRAS. In some embodiments, the cancer driver gene is PIK3CA. In some embodiments, the cancer driver gene is PTEN. In some embodiments, the cancer driver gene is H3F3A. In some embodiments, the cancer driver gene is KDR. In some embodiments, the cancer driver gene is MET.

Numerous EGFR mutations have been shown to be associated with lung cancer and are described in, for example: Morgensztern et al., J. Amer. Med. Assoc. Oncol, 2015, 1, 146-148; Bethune et al., J. Thorac. Dis., 2010, 2, 48-51; Kosaka et al., Cancer Res., 2004, 64, 8919-23; PCT Publication Nos. WO 2014/182521, WO 2012/156437, WO 2017/013160, and WO 2020/005932; and U.S. Patent Application Publications US 2018/0009782, US 2018/0216170, US 2019/0091205, and US 2015/0038520.

In some embodiments, the EGFR gene comprise mutations encoding T43A, E66*, L76P, N94D, S170R, L173P, N182S, C236R, V336M, V374A, N413T, F420L, L443P, V461A, R494G, 5498G, N603T, C628R, V651 A, G710S, K714R, G719S, G719C, G719A, E746V, L747S, E749Q, A750P, 1780V, T790M, R832C, A840T, L845R, L858R, L861Q, Y1016S, L1034R, and A1158P, or any combination thereof. In some embodiments, the EGFR gene comprises mutations in exon 18, exon 19, exon 20, or exon 21. For example, the EGFR exon 19 mutations can comprise E746-R748 deletion, E746-A750 deletion, E746-R748 deletion together with E749Q and A750P substitutions, del L747-E749 deletion combined with the A750P substitution, L747S substitution in combination with R748-P753 deletion, L747-S752 deletion in combination with E746V substitution, L747-T751 deletion combined with a serine insertion, AI insertion at positions M766-A767, an SVA insertion at positions S768-V769, or substitutions at position 719 in the nucleotide binding loop (exon 18) such as G719A, G719C, G710S, or any combination thereof. The EGFR exon 20 mutations can comprise one or more point mutations, insertions, and/or deletions of 3-18 nucleotides between amino acids 763-778. In some embodiments, EGFR exon 20 mutations can comprise mutations at one or more residues selected from the group consisting of A763, A767, S768, V769, D770, N771, P772, and H773. In some embodiments, the EGFR mutation can be at residue C797. In some embodiments, the mutations include substitution and/or deletion at the A763, A767, S768, V769, D770, N771, P772, and H773 in exon 20. In some embodiments, the exon 20 mutations are selected from the group consisting of A763insFQEA, A767insASV, S768dupSVD, V769insASV, D770insSVD, D770insNPG, H773insNPH, N771del insGY, N771del insFH, and N771dupNPH.

In some embodiments, the EGFR mutations can comprise a G719S mutation, a G719C mutation, a G719A mutation, an L861Q mutation, an L858R mutation, an L845R mutation, a T790M mutation, a C797S mutation, an ex19Del mutation, or an ex20Ins mutation, or any combination thereof.

In some embodiments, the EGFR gene comprises a deletion of exon 19, a point mutation in exon 21, a mutation in exon 18, or a mutation in exon 20, or any combination thereof. In some embodiments, the EGFR gene comprises a G719S mutation, a G719C mutation, a G719A mutation, an L861Q mutation, an L858R mutation, an L845R mutation, a T790M mutation, a C797S mutation, an ex19Del mutation, and an ex20Ins mutation, or any combination thereof.

In some embodiments, the human is a tobacco smoker. Estrogen hormones and their respective metabolites can be at elevated concentrations in lung tissue of a human because the human smokes tobacco products. In some embodiments, the human periodically smokes tobacco products, or has smoked tobacco products, or may smoke tobacco products. In some embodiments, the human may be (or have been) a light smoker. In some embodiments, the human may be (or have been) a moderate smoker. In some embodiments, the human may be (or have been) a heavy smoker. In some embodiments, the human is not a tobacco smoker. Tobacco products include, but are not limited to, cigarettes, cigars, pipes, hookahs, electronic nicotine delivery systems, and other forms in which tobacco leaves are burned and the resultant smoke inhaled. A Pack Year, typically calculated as the equivalent of a pack of cigarettes (20 cigarettes) per day for a year (including two packs of cigarettes per day for a half year, etc.) can be used as a measurement for the level of tobacco smoking in the human. In some embodiments, the human is a never-smoker (i.e., <100 cigarettes in a lifetime).

Estrogen hormones and their respective metabolites can also be at elevated concentrations in lung tissue of a human because the human is administered estrogen hormones, for example, as part of a hormone replacement therapy, because the human is pregnant, because the human is administered an estrogen-based contraceptive, because the human female has a reproductive disorder, or because the human male has a hormone-driven cancer. In addition, estrogen synthesis enzymes (e.g., aromatase) and precursors (e.g., testosterone) can be elevated such that higher levels of estrogen metabolites could be localized to where higher concentrations of the enzymes and/or precursors are present. In some embodiments, the human is female. In some embodiments, the human is male.

The lung cancer can be non-small cell lung cancer (NSCLC) or small cell lung cancer (SCLC). In some embodiments, the lung cancer is NSCLC. In some embodiments, the lung cancer is SCLC. NSCLC is further classified into adenocarcinoma, squamous cell carcinoma, and large cell carcinoma. Of these, adenocarcinoma is the most frequent subtype of NSCLC.

In some embodiments, the methods further comprise performing or having performed an assay on a biological sample obtained from the human, whereby the assay determines the activity of one or more enzymes in the metabolism of estrogen. In some embodiments, the one or more enzymes in the metabolism of estrogen is chosen from CYP1B1, CYP1A1, and COMT. In some embodiments, the enzyme in the metabolism of estrogen is CYP1B1. In some embodiments, the enzyme in the metabolism of estrogen is CYP1A1. In some embodiments, the enzyme in the metabolism of estrogen is COMT. When the human has a higher activity of one or more enzymes producing 4-OHEs compared to a cancer-free subject, then the human has a higher risk of developing the lung cancer event. When the human has the same or a higher activity of one or more enzymes producing 4-OMEs compared to a cancer-free subject, then the human has a lower risk of developing the lung cancer event. When the human has a higher activity of one or more enzymes producing 2-OHEs compared to a cancer-free subject, then the human has a higher risk of developing the lung cancer event. When the human has the same or a higher activity of one or more enzymes producing 2-OMEs compared to a cancer-free subject, then the human has a lower risk of developing the lung cancer event. The assay determining the activity of one or more enzymes in the metabolism of estrogen can be a biological assay used to determine the enzymatic activity of one or more enzymes in the metabolism of estrogen. Alternately, the assay determining the activity of one or more enzymes in the metabolism of estrogen can be a sequence analysis of the nucleic acid molecule encoding the one or more enzymes in the metabolism of estrogen. For example, a COMT 158 that has a GA+AA genotype is a low-activity allele. In addition, a COMT 158 that has a GG genotype is a high-activity allele. Variant isoforms of COMT are disclosed in, for example, Dawling et al., Cancer Res., 2001, 61, 6716-6722. Thus, the activity of 4-OHE-producing and/or 2-OHE-producing estrogen metabolism enzymes and/or the activity of 4-OME-producing and/or 2-OME-producing estrogen metabolism enzymes in the human can also be used for stratifying the risk of developing a lung cancer event.

In some embodiments, the methods described herein further comprise: administering to the human having a higher ratio of 4-OHEs to total estrogen, or a higher ratio of 2-OHEs to total estrogen, or a higher ratio of 4-OHEs to 2-OHEs (compared to a healthy subject, such as a cancer-free subject), a therapeutic agent that inhibits CYP1B1, a CYP1A1/CYP1B1 dual inhibitor, a therapeutic agent that induces or restores COMT, and/or a therapeutic agent that inhibits estrogen production; and/or administering to the human having an estrogen-induced mutation in the one or more cancer driver genes a therapeutic agent that inhibits the production or activity of the cancer driver gene product. In some embodiments, humans who are determined to be stratified at a high risk for developing a lung cancer event, such as by the methods described herein, can be selected for treatment.

Multiple inhibitors of CYP1B1 biological activity are described in, for example, Dutour and Poirier, Eur. J. Med. Chem., 2017, 135, 296-306 and Horley et al., Eur. J. Med. Chem., 2017, 129, 159-174. Suitable inhibitors of CYP1B1 (CYP1B1 inhibitors vs. known substrates) include, but are not limited to: stilbenes, such as resveratrol and its derivatives; polycyclic aromatic compounds, such as pyrene and its derivatives, 4-(1-propynyl)biphenyl, dibenz[a,c]anthracene, dibenz[a,h] anthracene and 3-methylcycloanthrene; flavonoids such as flavones, flavonols, dihydroflavonols, flavanones, flavanols, anthocyanidins, isoflavonoids, neoflavonoids and minor flavonoids (e.g. chalcones); coumarins, such as paradisin A, bergamottin and its derivatives, isopimpinellin, imperatorin, and isoimperatorin; anthraquinones, such as purpurin, alizarin, and emodin; alkaloids, such as rutaecarpine and its derivatives, berberine and its derivatives, palmatine, and jattorrhizine, thalifendine; as well as testosterone and 17β-estradiol (E2), cannabinol and its derivatives, 7-hydroxycymopolone, hydroxycymopochromanone, perillyl alcohol, pifithrin alpha, carnosol, metformin, melatonin, fluoxetine, erythromycin, and 2,3′,4,5′-Tetramethoxystilbene (TMS); 1-[(1E)-2-(3,5-dimethoxyphenyl)ethenyl]-2,4-dimethoxy-benzen). In some embodiments, the therapeutic agent that inhibits CYP1B1 is berberine, homoeriodictyol, 2,3′,4,5′-tetramethoxystilbene, or transresveratrol. In some embodiments, the therapeutic agent that inhibits CYP1B1 is berberine. In some embodiments, the therapeutic agent that inhibits CYP1B1 is homoeriodictyol. In some embodiments, the therapeutic agent that inhibits CYP1B1 is 2,3′,4,5′-tetramethoxystilbene. In some embodiments, the therapeutic agent that inhibits CYP1B1 is transresveratrol.

In some embodiments, the therapeutic agent that restores or induces COMT is a divalent metal cation, S-adenosylmethionine, indole-3-carcinol, diindolylmethane, or a phytoestrogen. In some embodiments, the therapeutic agent that restores or induces COMT is a divalent metal cation. In some embodiments, the therapeutic agent that restores or induces COMT is S-adenosylmethionine. In some embodiments, the therapeutic agent that restores or induces COMT is indole-3-carcinol. In some embodiments, the therapeutic agent that restores or induces COMT is diindolylmethane. In some embodiments, the therapeutic agent that restores or induces COMT is a phytoestrogen.

In some embodiments, the therapeutic agent that inhibits estrogen production is anastrozole, exemestane, or letrozole. In some embodiments, the therapeutic agent that inhibits estrogen production is anastrozole. In some embodiments, the therapeutic agent that inhibits estrogen production is exemestane. In some embodiments, the therapeutic agent that inhibits estrogen production is letrozole.

In some embodiments, the therapeutic agent that inhibits the production or activity of the cancer driver gene product is a tyrosine kinase inhibitor (TKI). In some embodiments, the TKI is INLYTA® (axitinib), SPRYCEL® (dasatinib), TARCEVA® (erlotinib), GLIVEC® (imatinib), TASIGNA® (nilotinib), VOTRIENT® (pazopanib), IRESSA® (gefitinib), GILOTRIF® or GIOTRIF® or AFANIX® (afatinib), TAGRISSO® or TAGRIX® (osimertinib), VIZIMPRO® (dacomitinib), or SUTENT® (sunitinib), or any combination thereof. In some embodiments, the TKI is INLYTA® (axitinib). In some embodiments, the TKI is SPRYCEL® (dasatinib). In some embodiments, the TKI is TARCEVA® (erlotinib). In some embodiments, the TKI is GLIVEC® (imatinib). In some embodiments, the TKI is TASIGNA® (nilotinib). In some embodiments, the TKI is VOTRIENT® (pazopanib). In some embodiments, the TKI is SUTENT® (sunitinib). In some embodiments, the TKI is IRESSA® (gefitinib). In some embodiments, the TKI is GILOTRIF® or GIOTRIF® or AFANIX® (afatinib). In some embodiments, the TKI is TAGRISSO® or TAGRIX® (osimertinib). In some embodiments, the TKI is VIZIMPRO® (dacomitinib). In some embodiments, the TKI is TARCEVA® (erlotinib), IRESSA® (gefitinib), GILOTRIF® or GIOTRIF® or AFANIX® (afatinib), TAGRISSO® or TAGRIX® (osimertinib), or VIZIMPRO® (dacomitinib), or any combination thereof. In some embodiments, the TKI is erlotinib, gefitinib, afatinib, osimertinib, or dacomitinib, or any combination thereof.

The methods described herein can be carried out at varying times in order to monitor treatment efficacy. For example, the methods, including any optional steps, can be repeated after a period of time, for example, as a way to monitor a human's health. Thus, for example, in some embodiments, the methods optionally further comprise repeating the assay, mutation analysis, and comparing steps after a period of time. Repeating the methods can be used, for example, to determine if a human has advanced from a healthy state to a precancerous or cancerous state. Repeating the methods can also be used, for example, to determine if the human's prognosis has improved based on a particular treatment regimen, or to determine if adjustments to the treatment regimen should be made to achieve improvement or to attain further improvement in the human's prognosis. The methods can be repeated at least one time, two times, three times, four times, or five or more times. The period of time between repeats can vary, and may be regular or irregular. In some embodiments, the methods are repeated in three-month intervals. In some embodiments, the methods are repeated in six-month intervals. In some embodiments, the methods are repeated in one-year intervals. In some embodiments, the methods are repeated in two-year intervals. In some embodiments, the methods are repeated in five-year intervals. In some embodiments, the methods are repeated only once, which may be about three months, six months, twelve months, eighteen months, two years, three years, four years, five years, or more from the initial assessment.

The methods described herein can also be used to identify a human having a lung cancer event who is susceptible to treatment with a therapeutic agent that inhibits CYP1B1, a CYP1A1/CYP1B1 dual inhibitor, a therapeutic agent that induces or restores COMT, a therapeutic agent that inhibits estrogen production, and/or a therapeutic agent that inhibits the production or activity of the cancer driver gene product. Without being bound by any particular theory or mechanism, it is believed, for example, that suppressing CYP1B1 mediated 4-OHE production (through, for example inhibiting the biological activity of CYP1B1) can impede tumorigenesis in lung cells, such as lung cells having a mutant EGFR.

The methods described herein can also be used to identify a human having a high risk of developing lung cancer (and who does not yet have lung cancer) who is susceptible to prophylactic treatment with a therapeutic agent that inhibits CYP1B1, a CYP1A1/CYP1B1 dual inhibitor, a therapeutic agent that induces or restores COMT, a therapeutic agent that inhibits estrogen production, and/or a therapeutic agent that inhibits the production or activity of the cancer driver gene product. Without being bound by any particular theory or mechanism, it is believed, for example, that suppressing CYP1B1 mediated 4-OHE production (through, for example inhibiting the biological activity of CYP1B1) can prevent tumorigenesis in lung cells.

The present disclosure also provides methods of treating a human having a high risk of developing a lung cancer event but who does not yet have lung cancer. In some embodiments, the method comprises administering to the human a therapeutic agent that inhibits CYP1B1, a CYP1A1/CYP1B1 dual inhibitor, a therapeutic agent that induces or restores COMT, and/or a therapeutic agent that inhibits estrogen production. In some embodiments, the therapeutic agent that inhibits CYP1B1 is 2,3′,4,5′-tetramethoxystilbene, berberine, homoeriodictyol, or transresveratrol. In some embodiments, the therapeutic agent that induces COMT is a divalent metal cation, S-adenosylmethionine, indole-3-carcinol, diindolylmethane, or a phytoestrogen. In some embodiments, the therapeutic agent that inhibits estrogen production is anastrozole, exemestane, or letrozole. In some embodiments, the methods further comprise administering to the human a tyrosine kinase inhibitor. In some embodiments, the tyrosine kinase inhibitor is erlotinib, gefitinib, afatinib, osimertinib, or dacomitinib, or any combination thereof. In some embodiments, the human at high risk of developing a lung cancer event has a higher ratio of 4-OHEs to total estrogen, or a higher ratio of 2-OHEs to total estrogen, or a higher ratio of 4-OHEs to 2-OHEs compared to the same ratios from a cancer-free subject. In some embodiments, the human at high risk of developing a lung cancer event has an estrogen-induced mutation in a lung cancer driver gene. In some embodiments, the human at high risk of developing a lung cancer event has an EGFR mutation.

In some embodiments, the human can be administered surgery, radiation therapy, proton therapy, ablation therapy, hormone therapy, hormone replacement therapy, chemotherapy, immunotherapy, stem cell therapy, follow up testing, diet management, vitamin supplementation, nutritional supplementation, exercise regimen, physical therapy, a prosthetic, transplantation, reconstruction, psychological counseling, social counseling, education, or regimen compliance management, or any combination thereof. In some embodiments, the human has also had surgery, chemotherapy, and/or immunotherapy.

Administration of any of the therapeutic agents described herein can be repeated, for example, after one day, two days, three days, five days, one week, two weeks, three weeks, one month, five weeks, six weeks, seven weeks, eight weeks, two months, or three months. The repeated administration can be at the same dose or at a different dose. The administration can be repeated once, twice, three times, four times, five times, six times, seven times, eight times, nine times, ten times, or more. For example, according to certain dosage regimens, a human can receive therapy for a prolonged period of time such as, for example, 6 months, 1 year, or more.

In some embodiments, a hormone replacement therapy regimen can be supplemented with one or more agents that inhibits CYP1B1, inhibits both CYP1A1 and CYP1B1, restores or induces COMT, and/or inhibits estrogen production. In some embodiments, a hormone replacement therapy regimen can be supplemented with one or more agents that inhibits CYP1B1. In some embodiments, a hormone replacement therapy regimen can be supplemented with one or more agents that induces or restores COMT. In some embodiments, a hormone replacement therapy regimen can be supplemented with one or more agents that inhibits estrogen production. Such agents are described herein.

Administration of any of the therapeutic agents described herein can occur by any suitable route including, but not limited to, parenteral, intravenous, oral, subcutaneous, intra-arterial, intracranial, intrathecal, transmucosal, intraperitoneal, topical, transdermal, intranasal, or intramuscular. Pharmaceutical compositions can be provided in unit dosage form (i.e., the dosage for a single administration). The therapeutic agents described herein can be administered in a manner compatible with the dosage formulation, and in a therapeutically effective amount according to the product labels. The treatment regimen can be tailored to the specific characteristics of the human, for example, the age, sex, or weight of the human, the type or stage of the cancer, and the overall health of the human.

The following examples are provided to describe the embodiments in greater detail. They are intended to illustrate, not to limit, the claimed embodiments. The following examples provide those of ordinary skill in the art with a disclosure and description of how the compounds, compositions, articles, devices and/or methods described herein are made and evaluated, and are intended to be purely exemplary and are not intended to limit the scope of any claims. Efforts have been made to ensure accuracy with respect to numbers (such as, for example, amounts, temperature, etc.), but some errors and deviations may be accounted for. Unless indicated otherwise, parts are parts by weight, temperature is in ° C. or is at ambient temperature, and pressure is at or near atmospheric.

### EXAMPLES

### Example 1

### Materials and Methods

### Treatment of Cell Lines

Three immortalized human bronchial epithelial cell lines (HBEC-57KT, -65KT, and -66KT) were used (see, Ramirez et al., Cancer Res., 2004, 64, 9027-34). For these cell lines, a normal phenotype and a near normal karyotype were retained—these cell lines do not grow in soft agar or form tumors in mice, and possess wild type EGFR and exhibit EGF-dependent growth (see, Gazdar et al., Lung Cancer., 2010, 68, 309-18). Cells were immortalized by over-expressing Cdk4 and human telomerase reverse transcriptase (hTERT) to emulate two of the earliest events in lung cancer pathogenesis: abrogation of the p16/RB cell cycle checkpoint pathway and bypass of replicative senescence. HBEC-57KT, -65KT, and -66KT cells were maintained in keratinocyte serum-free (KSF) media containing 50 μg/ml bovine pituitary extract and 5 ng/ml epidermal growth factor. Initially, cells were chronically treated with 0.1% DMSO (vehicle) or 100 nM E2, 4-OHE₁ or 4-OHE₂. Subsequently, a broader dose range was tested, including 2, 10, 50, or 100 nM E₂, 4-OHE₁, 4-OHE₂, 2-OHE₁, or 2-OHE₂. Exposure times ranged from a minimum of 18 weeks and a maximum of 40 weeks. An aliquot from each cell line and treatment was banked in liquid nitrogen every 1-2 weeks.

### Soft Agar Assay

At specific timepoints (18, 24 and 34 weeks post-treatment), each treated cell line was assayed for transformation by assessing anchorage independent growth in soft agar according to the method of de Larco and Todaro (see, de Larco et al., Proc. Natl. Acad. Sci. USA, 1978, 75, 4001-5). Briefly, each well of a 12-well plate was coated with 0.6% agarose/KSF media. Wells were overlaid with 0.3% agarose/KSF media containing 5000 HBEC-KT cells. RKO colon carcinoma cells were included as a positive control. Each assay was performed in triplicate. After 4-6 weeks, plates were either stained with crystal violet for counting or used to isolate viable clones. Transformed clones were picked using a p200 pipette tip, placed back in culture under pre-treatment conditions in KSF media, and expanded for future analyses. The genotypes of the cell lines used in these experiments are summarized in Table 1.

TABLE 1
Genotypes of HBEC-KT cells

| | HBEC57-KT | HBEC65-KT | HBEC-66KT |
|---|---|---|---|
| CYP1B1-48 | Arg/Gly | Arg/Arg | Arg/Gly |
| CYP1B1-432 | Leu/Val | Leu/Leu | Val/Val |
| CYP1B1-453 | Asn/Asn | Asn/Asn | Asn/Asn |
| COMT-158 | High/Low | High/Low | High/High |

### DNA Damage Assays

DNA was extracted from expanded clones for sequencing. The mutation status of 50 oncogenic driver genes was examined in transformed cells by next-gen sequencing using an AmpliSeq Illumina for Cancer Hotspot Panel v2. Data were analyzed using Illumina software. DNA damage induced by short-term treatment with estrogens was measured using the Comet assay (see, Mozaffarieh et al., Mol. Vis., 2008, 14, 1584-8). Other assays, however, may be used as desired.

### Analysis of Urinary Estrogen Species by UPLC-MS

A urinary method has been established (adapted from Xu et al., Nat. Protoc., 2007, 2, 1350-5) that utilizes deuterated estrogen metabolites as internal standards and employs enzymatic hydrolysis. UPLC-ESI-MS/MS was performed using a Water's I-class UPLC coupled to a Water's Xevo TQ-S micro tandem quadrupole mass spectrometer and a Water's Cortecs Phenyl column (40° C.) eluted using a linear gradient of solvent A (MeOH with 0.2 mM NH₄F) and solvent B (H₂O with 0.1% formic acid). The results were analyzed using MassLynx 4.2. A calibration curve was plotted (ratio of estrogen metabolite to its deuterated form) and the data fitted using linear regression with 1/×weighting. Metabolite levels were calculated as: response—intercept)/slope. Response is the ratio of the metabolite of interest to its deuterated internal standard. Slope and y-intercept were determined from the calibration curve.

### Genotyping

DNA was isolated from the buffy-coat of human blood samples using the Blood DNA isolation kit (Promega Inc., Madison, Wis.). Single nucleotide polymorphisms in the CYP1B1 (R48G/rs10012, L432V/rs1056836, and N453S/rs1800440) and COMT (V158M/rs4680) genes were genotyped by real-time PCR using TaqMan Genotyping Master Mix (ThermoFisher Scientific Inc.) according to the manufacturer's instructions. Results with >99% call rates and 100% concordance rates for duplicated specimens were subjected to further data analysis.

### Example 2

### Experimental Results

### Estrogen Metabolite Treatment Promotes Neoplastic Transformation in Bronchial Epithelial Cells

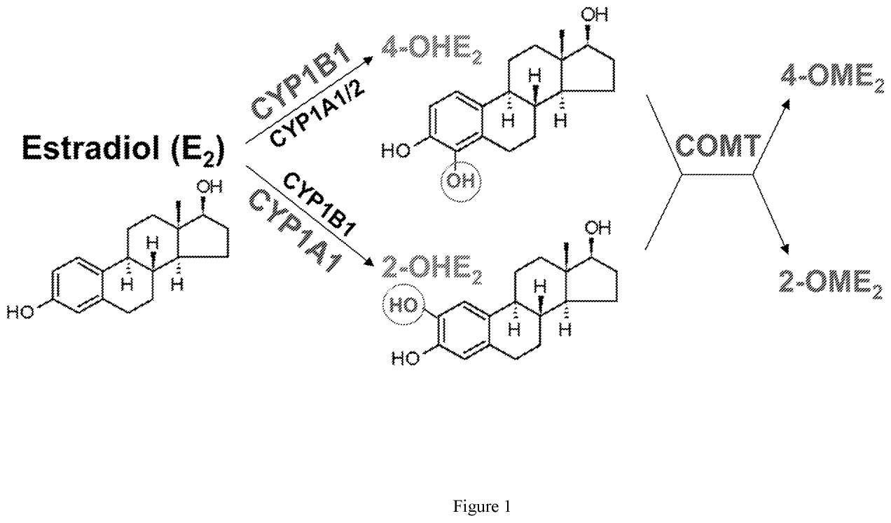

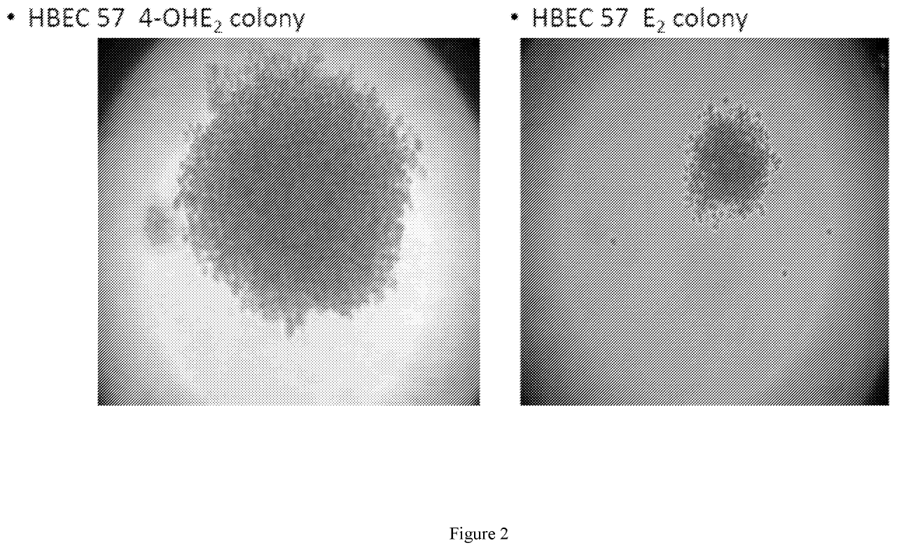

FIG. 2 shows exemplary crystal violet-stained HBEC-57KT colonies that grow in soft agar after treatment with 17β estradiol (E₂) or the estrogen metabolite 4-OHE₂ (see, FIG. 1). The results are summarized in Tables 2 and 3, and show that 35 weeks of chronic treatment with E₂ resulted in low level colony formation in both HBEC-57KT and HBEC-65KT cells while the 4-OHE₂ treatment resulted in efficient colony formation in HBEC-57KT, but not in HBEC-65KT cells or HBEC-66KT cells (having a low activity variant of CYP1B1 and high activity variant of COMT, the enzyme that inactivates detrimental 4-OHE, data not shown). 4-OHE₁ also transformed HBEC-57KT cells. None of the tested treatments (i.e., E₂, 4-OHE₁, or 4-OHE₂) transformed HBEC-66KT cells. Two transformed clones from each treatment group of HBEC-57KT cells were picked, expanded in the absence of estrogen treatment, and subjected to a second soft agar colony forming assay. These clones produced >100 colonies/well in soft agar, indicating that transformed HBEC-KTs are stable and can be maintained as a monolayer in vitro. Colonies from cells exposed for 18 weeks were diffuse and did not grow once picked (i.e., not fully transformed).

Clones of HBEC-KT 57 and 65 that had undergone estrogen-induced transformation were expanded in culture. Cells were harvested and DNA isolated for sequencing. Targeted sequencing was performed using the Illumina panel (AmpliSeq Illumina for Cancer HotSpot Panel v2) containing 50 oncogenic driver genes. Preliminary analysis of the resulting sequence data revealed the possible presence of somatic mutations in driver genes (Table 2 and Table 3).

TABLE 2
HBEC-57KT cells colony formation assay results
(two experiments shown)
HBEC-57KT-Experiment 2

| Treatment | # Colonies | # Picked | # Established |
|---|---|---|---|
| DMSO | 0 | 0 | 0 |
| E2 | 13 | 6 | 6 |
| 4-OHE₁ | 0 | 0 | 0 |
| 4-OHE₂ | 40 | 15 | 11 |

HBEC-57KT-Experiment 3

| Treatment | Weeks Treated | # Colonies |
|---|---|---|
| DMSO | 35 | 0 |
| E2 | 35 | 30 |
| 4-OHE₁ | 35 | 5 (small) |
| 4-OHE₂ | 35 | 54 |
| E2 | 18 | 0 |
| 4-OHE₂ | 18 | 4* |

*Colonies from cells exposed or 18 weeks were abnormal but diffuse and did not grow once picked (not fully transformed).

TABLE 3
HBEC-65KT cells colony formation assay results
(two experiments shown)
HBEC-65KT-Experiment 2

| Treatment | # Colonies | # Picked | # Established |
|---|---|---|---|
| DMSO | 0 | 0 | 0 |
| E2 | 11 | 6 | 4 |
| 4-OHE₁ | 0 | 0 | 0 |
| 4-OHE₂ | 0 | 0 | 0 |

HBEC-65KT-Experiment 3

| Treatment | Weeks Treated | # Colonies |
|---|---|---|
| DMSO | 35 | 0 |
| E2 | 35 | 34 |
| 4-OHE₁ | 35 | 0 |
| 4-OHE₂ | 35 | 0 |
| E2 | 18 | 0 |

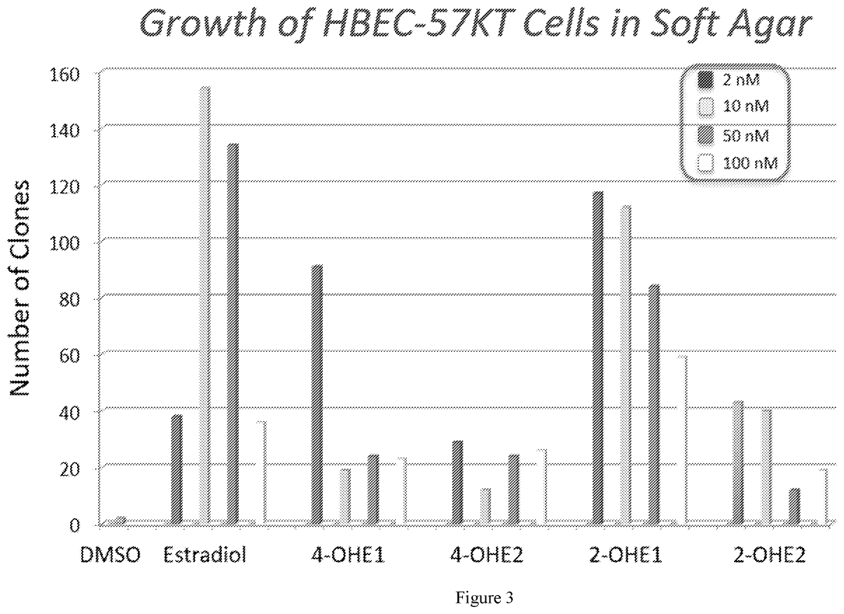

A subsequent experiment was performed to determine if concentrations of estrogen within the physiological range have the capability to transform HBECs. HBEC-57KT cells were treated chronically with 0.1% DMSO (vehicle) or 2, 10, 50, or 100 nM E2, 4-OHE₁, 4-OHE₂, 2-OHE₁ or 2-OHE₂ for 27 weeks. Transformation was determined by assessing the ability of the treated cells to grow in soft agar. FIG. 3 indicates that E2, 4-OHE₁, 4-OHE₂, 2-OHE₁ and 2-OHE₂ at 2, 10, 50, or 100 nM can transform HBEC-57KT cells.

### Estrogens Induce DNA Damage in HBEC-KT Lines

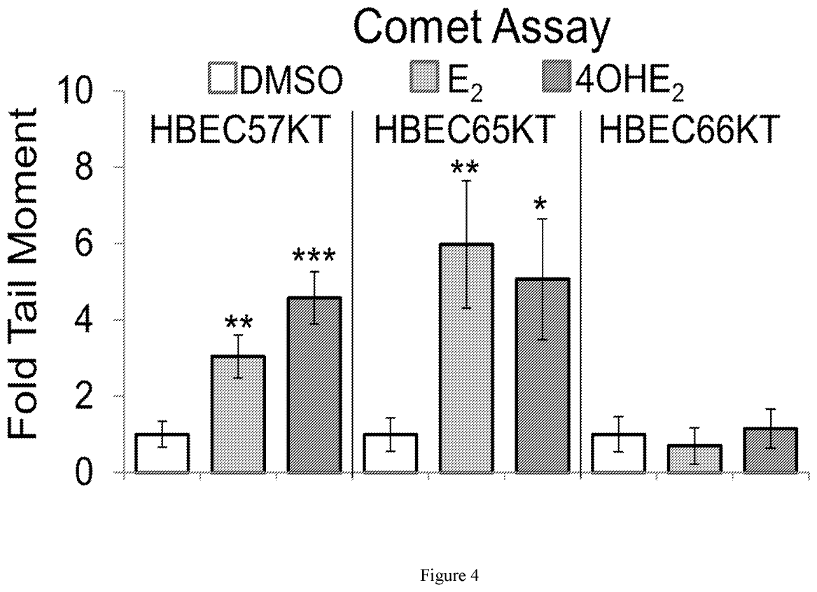

As shown in FIGS. 4, E₂ and 4-OHE₂ induce DNA damage in HBEC-KT cells with high-activity CYP1B1 and low-activity COMT (HBEC-57KT and -65KT). This profile of induced damage correlates with neoplastic transformation (HBEC-KT66 was negative for transformation by E₂, 4-OHE₁, and 4-OHE₂). Values=Fold difference of comet tail moment in E₂/4-OHE vs. DMSO-treated cells (set as 1) (mean±SEM). P≤0.05 (*); 0.01 (**) 0.001 (***) by t-test.

### Estrogen Metabolite Levels are Significantly Elevated in EGFR-Mutated NSCLC Patients

According to published reports, the two most common EGFR mutations are deletion of exon 19 and a point mutation in exon 21, which together account for approximately 90% of all EGFR mutations in NSCLC. Of the 14 patients with EGFR-mutated NSCLC in the present study sample, seven patients had tumors with mutations in EGFR exon 19 and five patients had mutations in exon 21. In the two remaining patients with uncommon mutations in EGFR, one had a mutation in exon 18 and the other had a mutation in exon 20. The EGFR mutation subtype of the seven NSCLC cases with the highest ratio of 4-OHE to 2-OHE (greater than 1.0) was six with mutations in exon 19 or 21, and one with a mutation in exon 18.

Advanced-stage NSCLC patients with EGFR- or ALK-mutated tumors and cancer-free subjects were examined. Cancer-free subjects were never-smoking women (see, Table 4). Urine specimens were collected and estrogen species (E₁, E₂, E₃, 4-OHEs, 2-OHEs, 2-OMEs) were quantified using UPLC-MS/MS. Estrogen metabolite levels (4-OHEs/total estrogen, 2-OHEs/total estrogen, 4-OHEs/2-OHEs) were compared between EGFR- and ALK-mutated NSCLC patients and cancer-free subjects (reference group).

TABLE 4
Study Sample for analysis of Urinary Estrogen Species by UPLC-MS

| | NSCLC cases | Cancer-free cases |
|---|---|---|
| | (n = 22) | (n = 17) |
| Gender | | |
| Female | 14 | 17 |
| Male | 8 | 0 |
| Median Age (min, max) | 69 (53-84) | 60 (50-70) |
| Smoking history | | |
| Never | 14 | 17 |
| Ever | 8 | 0 |
| Driver-mutation | | |
| EGFR | 14 | — |
| ALK | 8 | — |

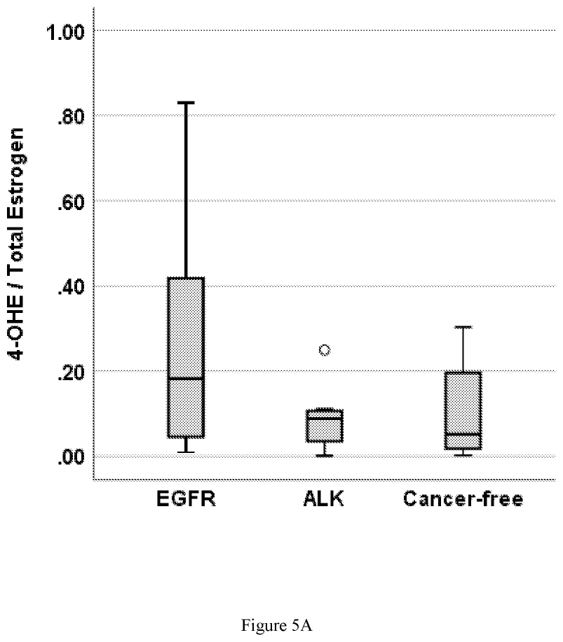

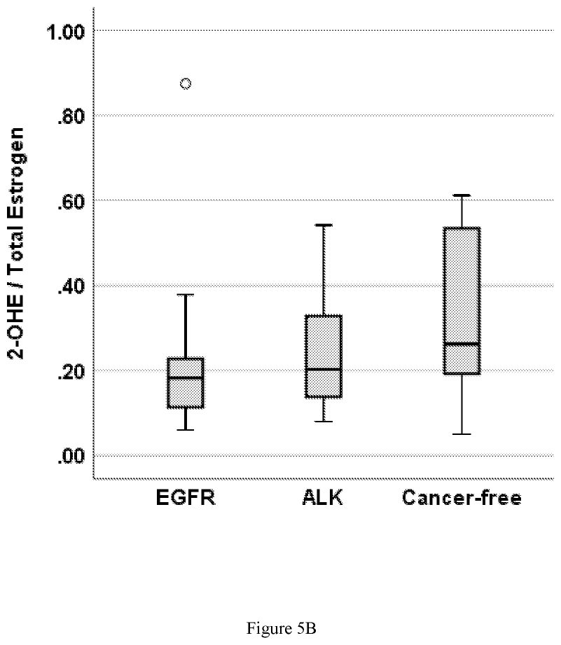

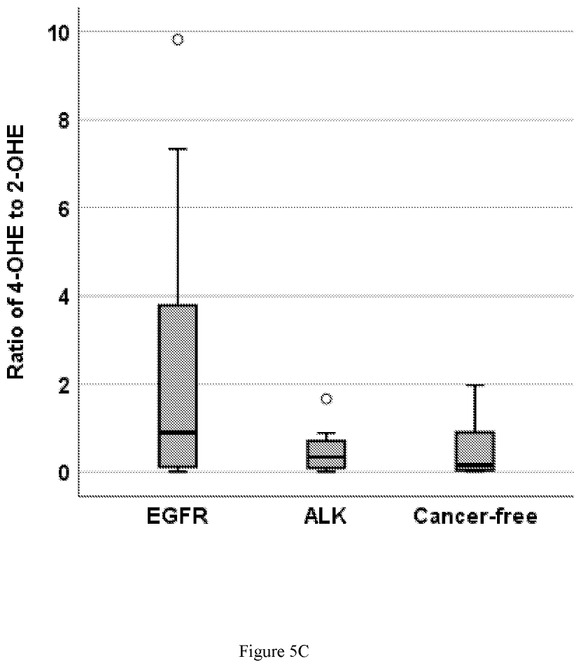

As shown in FIGS. 5A, 5B, and 5C, EGFR-mutated NSCLC patients had significantly higher 4-OHEs/total estrogen (0.18 vs. 0.05, p-value=0.048) and a trend towards lower 2-OHEs/total estrogen (0.18 vs. 0.26, p-value=0.084) compared to cancer-free subjects. The ratio of 4-OHEs/2-OHEs was higher in EGFR-mutated NSCLC patients compared to cancer-free subjects (0.90 vs. 0.16, p=0.053). In contrast, significant differences in estrogen metabolite measures were not observed between ALK-mutated NSCLC patients and cancer-free subjects. Greater 4-OHE to 2-OHE in EGFR-mutated NSCLC patients suggests production of 4-OHE is enhanced during EGFR-mutated lung tumorigenesis.

Genotypic analyses in a subset of the EGFR patients in the present study (n=11 with blood samples available) revealed an association between the presence of one or two low-activity alleles of COMT and higher 4-OHEs/2-OHEs as compared to subjects bearing two high activity alleles (median ratio 3.78 vs. 0.25). The results are shown in Table 5.

TABLE 5
Potential Correlation of Low Activity COMT Genotype with
High 4-OHE/2-OHE Ratio

| | | Median ratio | | |
|---|---|---|---|---|
| COMT 158 genotype | N | 4-OHEs/2-OHEs | Min | Max |
| GA + AA (1 or 2 low-activity alleles) | 7 | 3.78 | 0.07 | 9.82 |
| GG (2 high-activity alleles) | 4 | 0.25 | 0.01 | 1.18 |

Various modifications of the described subject matter, in addition to those described herein, will be apparent to those skilled in the art from the foregoing description. Such modifications are also intended to fall within the scope of the appended claims. Each reference (including, but not limited to, journal articles, U.S. and non-U.S. patents, patent application publications, international patent application publications, gene bank accession numbers, and the like) cited in the present application is incorporated herein by reference in its entirety.

What is claimed is:

1. A method for stratifying the risk of a human for developing a lung cancer event, the method comprising:
a) performing or having performed an assay on a biological sample obtained from the human, whereby the assay determines the level of total estrogen in the biological sample;
b) performing or having performed an assay on a biological sample obtained from the human, whereby the assay determines the level of 4-OHEs and/or 2-OHEs in the biological sample;
c) performing or having performed an analysis of estrogen-induced mutations in one or more cancer driver genes in a biological sample obtained from the human; and
d) determining the ratio of 4-OHEs to total estrogen, the ratio of 2-OHEs to total estrogen, and/or the ratio of 4-OHEs to 2-OHEs;
wherein the risk of developing a lung cancer event is stratified as:
i) when the human has an equal or lower ratio of 4-OHEs to total estrogen, or an equal or lower ratio of 2-OHEs to total estrogen, or an equal or lower ratio of 4-OHEs to 2-OHEs compared to the same ratios from a cancer-free subject, then the human has a lower risk of developing the lung cancer event;
ii) when the human has a higher ratio of 4-OHEs to total estrogen, or a higher ratio of 2-OHEs to total estrogen, or a higher ratio of 4-OHEs to 2-OHEs compared to the same ratios from a cancer-free subject, then the human has a higher risk of developing the lung cancer event; and
iii) when the human comprises an estrogen-induced mutation in the one or more cancer driver genes, then the human has a higher risk of developing the lung cancer event;
wherein the lung cancer event is developing a lung cancer, having a lung cancer recurrence, and/or a lower survival rate.

2. The method according to claim 1, wherein the level of 4-OHEs is the sum of 4-OHE₁ and 4-OHE₂.

3. The method according to claim 1 or claim 2, wherein the level of 2-OHEs is the sum of 2-OHE₁ and 2-OHE₂.

4. The method according to any one of claims 1 to 3, wherein the total estrogen is the sum of E₁, E₂, E₃, 4-OHE₁, 4-OHE₂, 2-OHE₁, 2-OHE₂, 2-OME₁, and 2-OME₂.

5. The method according to any one of claims 1 to 4, wherein the biological sample for determining the level of total estrogen, 4-OHEs, and/or 2-OHEs is urine, serum, or lung tissue.

6. The method according to any one of claims 1 to 5, wherein the biological sample for analysis of estrogen-induced mutations in one or more cancer driver genes is lung tissue or blood.

7. The method according to any one of claims 1 to 6, wherein the cancer driver gene having the estrogen-induced mutation is EGFR, ALK, ROS1, RET, BRAF, HER2, DDR2, FGFR1, PDGFRA, KRAS, PIK3CA, PTEN, H3F3A, KDR, or MET.

8. The method according to any one of claims 1 to 6, wherein the cancer driver gene having the estrogen-induced mutation is EGFR.

9. The method according to claim 8, wherein the EGFR gene comprises a deletion of exon 19 (ex19Del), a point mutation in exon 21, a mutation in exon 18, or a mutation in exon 20.

10. The method according to claim 8, wherein the EGFR gene comprises a G719S mutation, a G719C mutation, a G719A mutation, an L861Q mutation, an L858R mutation, an L845R mutation, a T790M mutation, a C797S mutation, an ex19Del mutation, or an ex20Ins mutation.

11. The method according to any one of claims 1 to 10, wherein the human is a tobacco smoker.

12. The method according to any one of claims 1 to 10, wherein the human is a never-smoker.

13. The method according to any one of claims 1 to 12, wherein the cancer is non-small cell lung cancer (NSCLC) or small cell lung cancer (SCLC).

14. The method according to any one of claims 1 to 12, wherein the cancer is NSCLC.

15. The method according to any one of claims 1 to 13, further comprising performing or having performed an assay on a biological sample obtained from the human, whereby the assay determines the activity of one or more enzymes in the metabolism of estrogen, wherein when the human has a higher activity of one or more enzymes producing 4-OHEs and/or 2-OHEs compared to a cancer-free subject, then the human has higher risk of developing the lung cancer event, and when the human has the same or a higher activity of one or more enzymes producing 2-OMEs and/or 4-OMEs compared to a cancer-free subject, then the human has a lower risk of developing the lung cancer event.

16. The method according to claim 15, wherein the one or more enzymes in the metabolism of estrogen is chosen from CYP1B1, CYP1A1, and COMT.

17. The method according to any one of claims 1 to 16, further comprising one or more of:
i) administering to the human having a higher ratio of 4-OHEs to total estrogen, or a higher ratio of 2-OHEs to total estrogen, or a higher ratio of 4-OHEs to 2-OHEs a therapeutic agent that inhibits CYP1B1, a CYP1A1/CYP1B1 dual inhibitor, a therapeutic agent that induces or restores COMT, and/or a therapeutic agent that inhibits estrogen production; and/or
ii) administering to the human having an estrogen-induced mutation in the one or more cancer driver genes a therapeutic agent that inhibits the production or activity of the cancer driver gene product.

18. The method according to claim 17, wherein the therapeutic agent that inhibits CYP1B1 is 2,3′,4,5′-tetramethoxystilbene, berberine, homoeriodictyol, or transresveratrol.

19. The method according to claim 17, wherein the therapeutic agent that induces COMT is a divalent metal cation, S-adenosylmethionine, indole-3-carcinol, diindolylmethane, or a phytoestrogen.

20. The method according to claim 17, wherein the therapeutic agent that inhibits estrogen production is anastrozole, exemestane, or letrozole.

21. The method according to claim 17, wherein the therapeutic agent that inhibits the production or activity of the cancer driver gene product is a tyrosine kinase inhibitor.

22. The method according to claim 21, wherein the tyrosine kinase inhibitor is erlotinib, gefitinib, afatinib, osimertinib, or dacomitinib, or any combination thereof.

23. The method according to claim 17, wherein the human is administered a combination of a tyrosine kinase inhibitor and one or more of a therapeutic agent that inhibits CYP1B1, a CYP1A1/CYP1B1 dual inhibitor, a therapeutic agent that induces or restores COMT, or a therapeutic agent that inhibits estrogen production.

24. The method according to any one of claims 1 to 23, wherein the human is administered surgery, radiation therapy, proton therapy, ablation therapy, hormone therapy, chemotherapy, immunotherapy, stem cell therapy, follow up testing, diet management, vitamin supplementation, nutritional supplementation, exercise regimen, physical therapy, a prosthetic, transplantation, reconstruction, psychological counseling, social counseling, education, or regimen compliance management, or any combination thereof.

25. A method of treating a human having a high risk of developing a lung cancer event but who does not yet have lung cancer, the method comprising administering to the human a therapeutic agent that inhibits CYP1B1, a CYP1A1/CYP1B1 dual inhibitor, a therapeutic agent that induces or restores COMT, or a therapeutic agent that inhibits estrogen production.

26. The method according to claim 25, wherein the therapeutic agent that inhibits CYP1B1 is 2,3′,4,5′-tetramethoxystilbene, berberine, homoeriodictyol, or transresveratrol.

27. The method according to claim 25, wherein the therapeutic agent that induces COMT is a divalent metal cation, S-adenosylmethionine, indole-3-carcinol, diindolylmethane, or a phytoestrogen.

28. The method according to claim 25, wherein the therapeutic agent that inhibits estrogen production is anastrozole, exemestane, or letrozole.

29. The method according to any one of claims 25 to 28, further comprising administering to the human a tyrosine kinase inhibitor.

30. The method according to claim 29, wherein the tyrosine kinase inhibitor is erlotinib, gefitinib, afatinib, osimertinib, or dacomitinib, or any combination thereof.

31. The method according to any one of claims 25 to 30, wherein the human has an EGFR mutation.
